SEQUENCE LISTING (10 sequences)

SEQ ID NO: 1 - DNA, 579 bp, Homo sapiens
atggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaatgaatggacactac

SEQ ID NO: 2 - DNA, 246 bp, Homo sapiens
atgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtac

SEQ ID NO: 3 - DNA, 621 bp, Homo sapiens
atgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctga

SEQ ID NO: 4 - protein, 8 aa, Artificial Sequence
  Primer
REKRAVVG

SEQ ID NO: 5 - DNA, 30 bp, Artificial Sequence
  Primer
attgaaagcttatggaaaacagatggcagg

SEQ ID NO: 6 - DNA, 36 bp, Artificial Sequence
  Primer
tactattataggttgcatctcgtgtccattcattgt

SEQ ID NO: 7 - DNA, 30 bp, Artificial Sequence
  Primer
ggacacgagatgcaacctataatagtagca

SEQ ID NO: 8 - DNA, 42 bp, Artificial Sequence
  Primer
tgaccacttgccacccatctcgagatcatcaatatcccaagg

SEQ ID NO: 9 - DNA, 28 bp, Artificial Sequence
  Primer
ggagatgggtggcaagtggtcaaaaagt

SEQ ID NO: 10 - DNA, 30 bp, Artificial Sequence
  Primer
cgcaagcttcgatgtcagcagtctttgtag

(19) United States
(12) Patent Application Publication
(10) Pub. No.: US 2004/0223974 A1
(43) Pub. Date: Nov. 11, 2004

(54) Vaccines, immunotherapeutics and methods for using the same

(76) Inventors: David B. Weiner, Merion Station, PA (US); Jeong-Im Sin, Seoul (KR)

Correspondence Address: COZEN O'CONNOR, P.C., 1900 MARKET STREET, PHILADELPHIA, PA, 19103-3508, US

(21) Appl. No.: 10/276,050

(22) PCT Filed: May 14, 2001
(86) PCT No.: PCT/US01/15600

Related U.S. Application Data
(60) Provisional application No. 60/203,601, filed on May 12, 2000.

(51) Int. Cl. A61K 39/00; A61K 39/38

(52) U.S. Cl. 424/184.1

(57) ABSTRACT

Compositions comprising isolated RANTES protein and/or a nucleic acid molecule that encodes RANTES protein in combination with isolated IL-8 protein and/or a nucleic acid molecule that encodes IL-8 protein and optionally further comprising an immunogen and/or a nucleic acid molecule that encodes an immunogen are disclosed. Methods of inducing an immune response in an individual against an immunogen comprising administering to the individual isolated RANTES protein and/or a nucleic acid molecule that encodes RANTES protein in combination with isolated IL-8 protein and/or a nucleic acid molecule that encodes IL-8 protein and additionally a target protein and/or a nucleic acid molecule that encodes a target protein are disclosed. Methods of modulating an individual's immune system comprising administering to the individual isolated RANTES protein and/or a nucleic acid molecule that encodes RANTES protein in combination with isolated IL-8 protein and/or a nucleic acid molecule that encodes IL-8 protein are also disclosed. In addition, recombinant vaccines, live attenuated pathogens comprising a nucleotide sequence that encodes IL-8 and a nucleotide sequence that encodes RANTES and methods of the same are disclosed.

### FIELD OF THE INVENTION

The present invention relates to improved vaccines, improved methods for prophylactically and/or therapeutically immunizing individuals against immunogens, and to improved immunotherapeutic compositions and improved immunotherapy methods.

### BACKGROUND OF THE INVENTION

Immunotherapy refers to modulating a person's immune responses to impart a desirable therapeutic effect. Immunotherapeutics refer to those compositions which, when administered to an individual, modulate the individual's immune system sufficient to ultimately decrease symptoms which are associated with undesirable immune responses or to ultimately alleviate symptoms by increasing desirable immune responses.

In some cases, immunotherapy is part of a vaccination protocol in which the individual is administered a vaccine that exposes the individual to an immunogen against which the individual generates an immune response. In such cases, the immunotherapeutic increases the immune response and/or selectively enhances a portion of the immune response (such as the cellular arm or the humoral arm) which is desirable to treat or prevent the particular condition, infection or disease.

In some cases, immunotherapeutics are delivered free of immunogens. In such cases, the immunotherapeutics are provided to modulate the immune system by either decreasing or suppressing immune responses, enhancing or increasing immune responses, decreasing or suppressing a portion of immune system, or enhancing or increasing a portion of the immune system.

In some cases, immunotherapeutics include antibodies which, when administered in vivo, bind to proteins involved in modulating immune responses. The interaction between antibodies and proteins involved in modulating immune responses results in the alteration of immune responses in the individual. For example, if the protein is involved in autoimmune disease, the antibodies can inhibit its activity in that role and reduce or eliminate the symptoms or disease.

Vaccines are useful to immunize individuals against target antigens such as allergens, pathogen antigens or antigens associated with cells involved in human diseases. Antigens associated with cells involved in human diseases include cancer-associated tumor antigens and antigens associated with cells involved in autoimmune diseases.

In designing such vaccines, it has been recognized that vaccines which produce the target antigen in cells of the vaccinated individual are effective in inducing the cellular arm of the immune system. Specifically, live attenuated vaccines, recombinant vaccines which use avirulent vectors, and DNA vaccines each lead to the production of antigens in the cell of the vaccinated individual which results in induction of the cellular arm of the immune system. On the other hand, killed or inactivated vaccines, and sub-unit vaccines which comprise only proteins do not induce good cellular immune responses although they do induce a humoral response.

A cellular immune response is often necessary to provide protection against pathogen infection and to provide effective immune-mediated therapy for treatment of pathogen infection, cancer or autoimmune diseases. Accordingly, vaccines which produce the target antigen in cells of the vaccinated individual such as live attenuated vaccines, recombinant vaccines which use avirulent vectors and DNA vaccines are often preferred.

While such vaccines are often effective to immunize individuals prophylactically or therapeutically against pathogen infection or human diseases, there is a need for improved vaccines. There is a need for compositions and methods which produce an enhanced immune response.

Likewise, while some immunotherapeutics are useful to modulate immune response in a patient, there remains a need for improved immunotherapeutic compositions and methods.

### SUMMARY OF THE INVENTION

The present invention relates to a composition comprising isolated RANTES protein and/or a nucleic acid molecule that encodes RANTES protein in combination with isolated IL-8 protein and/or a nucleic acid molecule that encodes IL-8 protein.

The present invention further relates to a composition comprising isolated RANTES protein and/or a nucleic acid molecule that encodes RANTES protein in combination with isolated IL-8 protein and/or a nucleic acid molecule that encodes IL-8 protein and further comprising a target protein and/or a nucleic acid molecule that encodes a target protein.

The present invention relates to injectable pharmaceutical compositions comprising isolated RANTES protein and/or a nucleic acid molecule that encodes RANTES protein in combination with isolated IL-8 protein and/or a nucleic acid molecule that encodes IL-8 protein.

The present invention relates to injectable pharmaceutical compositions comprising isolated RANTES protein and/or a nucleic acid molecule that encodes RANTES protein in combination with isolated IL-8 protein and/or a nucleic acid molecule that encodes IL-8 protein and further comprising a target protein and/or a nucleic acid molecule that encodes a target protein.

The present invention further relates to methods of inducing an immune response in an individual against an immunogen comprising administering to the individual isolated RANTES protein and/or a nucleic acid molecule that encodes RANTES protein in combination with isolated IL-8 protein and/or a nucleic acid molecule that encodes IL-8 protein and additionally a target protein and/or a nucleic acid molecule that encodes a target protein.

The present invention further relates to methods of modulating an individual's immune system comprising administering to the individual isolated RANTES protein and/or a nucleic acid molecule that encodes RANTES protein in combination with isolated IL-8 protein and/or a nucleic acid molecule that encodes IL-8 protein.

The present invention further relates to recombinant vaccines comprising a nucleotide sequence that encodes an immunogen operably linked to regulatory elements, a nucleotide sequence that encodes IL-8, and a nucleotide sequence that encodes RANTES, and to methods of inducing an immune response in an individual against an immunogen comprising administering such a recombinant vaccine to an individual.

The present invention further relates to a live attenuated pathogen comprising a nucleotide sequence that encodes IL-8 and a nucleotide sequence that encodes RANTES and to methods of inducing an immune response in an individual against a pathogen comprising administering the live attenuated pathogen to an individual.

### BRIEF DESCRIPTION OF THE DRAWINGS

FIG. 1 shows levels of systemic gD-specific in mice (Balb/c) immunized with DNA vectors in experiments described in the Example. Each group of mice (n=10) was immunized with gD DNA vaccines (60 μg per mouse) plus chemokine genes (40 μg per mouse) or TNF genes (40 μg per mouse) at 0 and 2 weeks. The mice were bled 2 weeks after the second immunization, and then equally pooled sera per group were serially diluted for reaction with gD. The ELISA titers were determined as the reverse of the highest sera dilution showing the same optical density as sera of naive mice. The absorbance (O.D.) Was measured at 405 nm.

FIGS. 2A and 2B show levels of IgG subclass in mice (Balb/c) immunized with DNA vectors in experiments described in the Example. In FIG. 2A, each group of mice (n=10) was immunized with gD DNA vaccines (60 μg per mouse) plus chemokine genes (40 μg per mouse) of TNF genes (40 μg per mouser) at 0 and 2 weeks. The mice were bled 2 weeks after the last immunization and then sera were diluted to 1:100 for reaction with gD. The absorbance (O.D.) Was measured at 405 nm. The relative optical density was calculated as optical density of each IgG subclass/total optical density. Line bars represent the mean (n=10) of relative optical densities of each mouse IgG subclass. FIG. 2B shows the relative ratio of IgG2a to IgG1. The mean (n=10) IgG2a level was divided by the mean IgG1 level in each immunization group. *Statistically significant at P<0.05 using Student's t test compared to each corresponding isotype of gD DN vaccine alone.

FIGS. 3A, 3B and 3C show Th-cell proliferation levels of splenocytes after in vitro gD stimulation in mice (Balb/c) coimmunized with α chemokine cDNA (FIG. 3A), β chemokine cDNA (FIG. 3B) and the TNF controls (FIG. 3C) in experiments described in the Example. Each group of mice (n=2) was immunized with gD DNA vaccines (60 μg per mouse) plus chemokine genes (40 μg per mouse) or TNF genes (40 μg per mouse) at 0 and 2 weeks. Two weeks after the last DNA injection, two mice were sacrificed and spleen cells were pooled for the proliferation assay. Splenocytes were stimulated with 1 and 5 μg of a gD-2 proteins per ml and 5 μf of PHA per ml as a positive control. After 3 days of stimulation, the cells were harvested and the cpm was counted. Samples were assayed in triplicate. The figures show the results of one of three separate experiments with similar results. The PHA control sample showed a stimulation index of 40-50. *Statistically significant at P<0.05 using Student's t teat compared to gD DNA vaccine alone.

FIGS. 4A, 4B and 4C show survival rates of mice (Balb/c) immunized with gD DNA vaccines plus α chemokine cDNA (FIG. 4A), β chemokine cDNA (FIG. 4B) and the TNF controls (FIG. 4C) in experiments described in the Example. Each group of mice (n=8) was immunized with gD DNA vaccines (60 μg per mouse) plus chemokine genes (40 μg per mouse) or TNF genes (40 μg per mouse) at 0 and 2 weeks. Three weeks after the second immunization, the mice were challenged i. vag. With 200 LD₅₀ of HSV-2 strain 186 (7×10⁵ PFU). Mice were then examined daily to evaluate survival rates. Surviving mice were counted for 61 days following viral challenge. This was repeated once with the expected results.

FIG. 5 shows the difference in protection rates between chemokine coinjections in experiments described in the Examples. Numbers in parentheses are the number of surviving animals/number tested in total.

### DETAILED DESCRIPTION OF PREFERRED EMBODIMENTS

As used herein, the term “immunomodulating proteins” is meant to refer to RANTES protein and IL-8 protein.

As used herein the term “target protein” is meant to refer to peptides and protein encoded by gene constructs of the present invention which act as target proteins for an immune response. The terms “target protein” and “immunogen” are used interchangeably and refer to a protein against which an immune response can be elicited. The target protein is an immunogenic protein which shares at least an epitope with a protein from the pathogen or undesirable cell-type such as a cancer cell or a cell involved in autoimmune disease against which an immune response is desired. The immune response directed against the target protein will protect the individual against and/or treat the individual for the specific infection or disease with which the target protein is associated.

As used herein, the term “genetic construct” refers to the DNA or RNA molecules that comprise a nucleotide sequence which encodes a target protein or immunomodulating protein. The coding sequence includes initiation and termination signals operably linked to regulatory elements including a promoter and polyadenylation signal capable of directing expression in the cells of the individual to whom the nucleic acid molecule is administered.

As used herein, the term “expressible form” refers to gene constructs which contain the necessary regulatory elements operable linked to a coding sequence that encodes a target protein or an immunomodulating protein, such that when present in the cell of the individual, the coding sequence will be expressed.

As used herein, the term “sharing an epitope” refers to proteins which comprise at least one epitope that is identical to or substantially similar to an epitope of another protein.

As used herein, the term “substantially similar epitope” is meant to refer to an epitope that has a structure which is not identical to an epitope of a protein but nonetheless invokes an cellular or humoral immune response which cross reacts to that protein.

As used herein, the term “intracellular pathogen” is meant to refer to a virus or pathogenic organism that, at least part of its reproductive or life cycle, exists within a host cell and therein produces or causes to be produced, pathogen proteins.

As used herein, the term “hyperproliferative diseases” is meant to refer to those diseases and disorders characterized by hyperproliferation of cells.

As used herein, the term “hyperproliferative-associated protein” is meant to refer to proteins that are associated with a hyperproliferative disease.

The invention arises from the discovery that a combination of IL-8 and RANTES modulates immune responses. Accordingly, a combination of these proteins and/or nucleic acid molecules encoding these proteins may be delivered as immunotherapeutics, or in combination with or as components of a vaccine. The combination of RANTES and IL-8 has been found to drive antigen specific Th1-type immune responses and enhance protective immunity when administered as part of a vaccine.

Immunomodulating proteins that induce and enhance CTL responses are particularly useful when administered in conjunction or as part of a vaccine against an intracellular pathogens, or against cells associated with autoimmune disease or cancer. Immunomodulating proteins that induce and enhance CTL responses are particularly useful when administered in conjunction with live attenuated vaccines, cell vaccines, recombinant vaccines, and nucleic acid/DNA vaccines. Alternatively, immunomodulating proteins that induce and enhance CTL responses are useful as immunotherapeutics which are administered to patients suffering from cancer or intracellular infection. Immunomodulating proteins that induce and enhance CTL responses are useful when administered to immunocompromised patients.

Immunomodulating proteins that induce and enhance T cell proliferation responses are particularly useful when administered in conjunction or as part of vaccines. Alternatively, immunomodulating proteins that induce and enhance T cell proliferation responses are useful as immunotherapeutics. Immunomodulating proteins that induce and enhance T cell proliferation responses are useful when administered to immunocompromised patients.

The GENBANK Accession number for the nucleotide and amino acid sequences for RANTES is M21121, which is incorporated herein by reference. RANTES is described in Schall, T. J., et al., J. Immunol. 141, 1018-1025 (1988), which is incorporated herein by reference.

The GENBANK Accession number for the nucleotide and amino acid sequences for IL-8 is M28130, which is incorporated herein by reference. IL-8 is described in Mukaida, N., et al., J. Immunol. 143(4), 1366-1371 (1989), which is incorporated herein by reference.

According to some embodiments of the invention, the combination of IL-8 and RANTES is delivered to an individual to modulate the activity of the individual's immune system. The IL-8 and RANTES may each, independently, be delivered directly in protein form and/or as nucleic acid molecules which comprise nucleotide sequences that encode the protein operably linked to regulatory elements necessary for expression in the individual. When the nucleic acid molecules are taken up by cells of the individual, the nucleotide sequences that encode the protein are expressed in the cells and the protein is thereby delivered to the individual. Aspects of the invention provide methods of delivering IL-8 protein and/or a nucleic acid molecule that encodes IL-8 in combination with RANTES protein and/or a nucleic acid molecule that encodes RANTES and compositions for delivering the same. Accordingly, some embodiments of the invention relate to combinations that comprise RANTES protein and/or nucleic acid molecules that encode RANTES protein and IL-8 protein and/or nucleic acid molecules that encode IL-8 protein. According to some embodiments, the compositions comprise combinations selected from the group consisting of: 1) RANTES protein and IL-8 protein; 2) nucleic acid molecules that encode RANTES protein and nucleic acid molecules that encode IL-8 protein; 3) RANTES protein and nucleic acid molecules that encode IL-8 protein; 4) IL-8 protein and nucleic acid molecules that encode RANTES protein; 5) RANTES protein, IL-8 protein and nucleic acid molecules that encode IL-8 protein; 6) RANTES protein and IL-8 protein and nucleic acid molecules that encode RANTES protein; 7) RANTES protein and nucleic acid molecules that encode RANTES protein and nucleic acid molecules that encode IL-8 protein; 8) IL-8 protein and/or nucleic acid molecules that encode RANTES protein and nucleic acid molecules that encode IL-8 protein; and 9) RANTES protein and nucleic acid molecules that encode RANTES protein and IL-8 protein and nucleic acid molecules that encode IL-8 protein.

According to some embodiments of the invention, the combination of IL-8 and RANTES delivered to an individual to modulate the activity of the individual's immune system is administered in combination with a vaccine. According to some aspects of the present invention, compositions and methods are provided which prophylactically and/or therapeutically immunize an individual against a pathogen or abnormal, disease-related cells. The IL-8 and RANTES may each, independently, be delivered directly in protein form and/or as nucleic acid molecules which comprise nucleotide sequences that encode the protein operably linked to regulatory elements necessary for expression in the individual. When the nucleic acid molecules are taken up by cells of the individual, the nucleotide sequences that encode the protein are expressed in the cells and the protein is thereby delivered to the individual. The vaccine may be any type of vaccine such as, for example, a subunit or protein vaccine, a killed or inactivated vaccine, a live attenuated vaccine, a cell vaccine, a recombinant vaccine or a nucleic acid or DNA vaccine. In the case of a live attenuated vaccines, a cell vaccine, a recombinant vaccine or a nucleic acid or DNA vaccine, the RANTES protein and or the IL-8 protein may be encoded by the nucleic acid molecules of these vaccines. By delivering IL-8 protein and/or a nucleic acid molecule that encodes IL-8 in combination with RANTES protein and/or a nucleic acid molecule that encodes RANTES, the immune response induced by the vaccine may be modulated, particularly by enhancing the cellular arm According to some embodiments, the compositions comprise vaccines such as a protein vaccine and/or a killed vaccine and/or an inactivated vaccine and/or a live attenuated vaccine and/or a recombinant vaccine and/or a DNA vaccine in combination with and/or otherwise including a combination selected from the group consisting of: 1) RANTES protein and IL-8 protein; 2) nucleic acid molecules that encode RANTES protein and nucleic acid molecules that encode IL-8 protein; 3) RANTES protein and nucleic acid molecules that encode IL-8 protein; 4) IL-8 protein and nucleic acid molecules that encode RANTES protein; 5) RANTES protein, IL-8 protein and nucleic acid molecules that encode IL-8 protein; 6) RANTES protein and IL-8 protein and nucleic acid molecules that encode RANTES protein; 7) RANTES protein and nucleic acid molecules that encode RANTES protein and nucleic acid molecules that encode IL-8 protein; 8) IL-8 protein and/or nucleic acid molecules that encode RANTES protein and nucleic acid molecules that encode IL-8 protein; and 9) RANTES protein and nucleic acid molecules that encode RANTES protein and IL-8 protein and nucleic acid molecules that encode IL-8 protein.

Below is a description of the preparation and administration of immunomodulating proteins in protein form followed by a description of the preparation and administration of nucleic acid molecules that encode immunomodulating proteins. As discussed herein, compositions and methods of the invention can include combinations of proteins and nucleic acids as well as compositions and methods which include only proteins and compositions and methods which include only nucleic acids. Accordingly, the description set for below is intended to include compositions and methods which include the use of combinations of proteins and nucleic acids.

As noted above, RANTES protein and/or IL-8 protein may be administered as part of an immunotherapy and/or vaccine protocol in order to modulate immune responses. The immunomodulating proteins may be prepared by recombinant methodology, synthesized by standard protein synthesis techniques or isolated and purified from natural sources. Hybridomas which produce antibodies that bind to the protein can be generated and used in isolation and purification procedures. cDNAs that encode this protein have been isolated, sequenced, incorporated into vectors including expression vector which were introduced into host cells that then express the proteins recombinantly.

Isolated cDNA that encodes either of the immunomodulating proteins is useful as a starting material in the construction of recombinant expression vectors that can produce that immunomodulating protein. The cDNA is incorporated into vectors including expression vectors which are introduced into host cells that then express the proteins recombinantly.

Using standard techniques and readily available starting materials, a nucleic acid molecule that encodes an immunomodulating protein may be prepared. The nucleic acid molecule may be incorporated into an expression vector which is then incorporated into a host cell. Host cells for use in well known recombinant expression systems for production of proteins are well known and readily available. Examples of host cells include bacteria cells such as E. coli, yeast cells such as S. cerevisiae, insect cells such as S. frugiperda, non-human mammalian tissue culture cells Chinese hamster ovary (CHO) cells and human tissue culture cells such as HeLa cells.

In some embodiments, for example, one having ordinary skill in the art can, using well known techniques, insert DNA molecules into a commercially available expression vector for use in well known expression systems. For example, the commercially available plasmid pSE420 (Invitrogen, San Diego, Calif.) may be used for production of immunomodulating proteins in E. coli. The commercially available plasmid pYES2 (Invitrogen, San Diego, Calif.) may, for example, be used for production in S. cerevisiae strains of yeast. The commercially available MAXBAC™ complete baculovirus expression system (Invitrogen, San Diego, Calif.) may, for example, be used for production in insect cells. The commercially available plasmid pcDNA I or pcDNA3 (Invitrogen, San Diego, Calif.) may, for example, be used for production in mammalian cells such as Chinese Hamster Ovary cells. One having ordinary skill in the art can use these commercial expression vectors and systems or others to produce immunomodulating proteins by routine techniques and readily available starting materials. (See e.g., Sambrook et al., Molecular Cloning a Laboratory Manual, Second Ed. Cold Spring Harbor Press (1989) which is incorporated herein by reference.) Thus, the desired proteins can be prepared in both prokaryotic and eukaryotic systems, resulting in a spectrum of processed forms of the protein.

One having ordinary skill in the art may use other commercially available expression vectors and systems or produce vectors using well known methods and readily available starting materials. Expression systems containing the requisite control sequences, such as promoters and polyadenylation signals, and preferably enhancers, are readily available and known in the art for a variety of hosts. See e.g., Sambrook et al., Molecular Cloning a Laboratory Manual, Second Ed. Cold Spring Harbor Press (1989).

The expression vector including the DNA that encodes an immunomodulating protein is used to transform the compatible host which is then cultured and maintained under conditions wherein expression of the foreign DNA takes place. The protein of the present invention thus produced is recovered from the culture, either by lysing the cells or from the culture medium as appropriate and known to those in the art. One having ordinary skill in the art can, using well known techniques, isolate the immunomodulating protein that is produced using such expression systems. The methods of purifying proteins from natural sources using antibodies may be equally applied to purifying protein produced by recombinant DNA methodology.

The immunomodulating protein(s) can be formulated into pharmaceutical compositions. Suitable pharmaceutical carriers are described in Remmington's Pharmaceutical Sciences, A. Osol, a standard reference text in this field, which is incorporated herein by reference. The pharmaceutical compositions of the present invention may be administered by any means that enables the active agent to reach the targeted cells. Because peptides are subject to being digested when administered orally, parenteral administration, i.e., intravenous, subcutaneous, transdermal, intramuscular, would ordinarily be used to optimize absorption. Intravenous administration may be accomplished with the aid of an infusion pump. The pharmaceutical compositions of the present invention may be formulated as an emulsion. Alternatively, they may be formulated as aerosol medicaments for intranasal or inhalation administration. In some cases, topical administration may be desirable.

The dosage administered varies depending upon factors such as: pharmacodynamic characteristics; its mode and route of administration; age, health, and weight of the recipient; nature and extent of symptoms; kind of concurrent treatment; and frequency of treatment. Usually, the dosage of protein can be about 1 to 3000 milligrams per 50 kilograms of body weight; preferably 10 to 1000 milligrams per 50 kilograms of body weight; more preferably 25 to 800 milligrams per 50 kilograms of body weight. Ordinarily 8 to 800 milligrams are administered to an individual per day in divided doses 1 to 6 times a day or in sustained release form is effective to obtain desired results. Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Compositions for oral administration include powders or granules, suspensions or solutions in water or non-aqueous media, capsules, sachets or tablets. Thickeners, flavoring agents, diluents, emulsifiers, dispersing aids or binders may be desirable. Compositions for parenteral, intravenous, intrathecal or intraventricular administration may include sterile aqueous solutions which may also contain buffers, diluents and other suitable additives and are preferably sterile and pyrogen free. Pharmaceutical compositions which are suitable for intravenous administration according to the invention are sterile and pyrogen free.

For parenteral administration, proteins can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. For example, a parenteral composition suitable for administration by injection is prepared by dissolving 1.5% by weight of active ingredient in 0.9% sodium chloride solution.

The pharmaceutical compositions of the present invention may be administered by any means that enables the active agent to reach the agent's site of action in the body of a mammal. The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, pulmonary administration, e.g., by inhalation or insufflation, or intrathecal or intraventricular administration.

In some embodiments, IL-8 and/or RANTES proteins are each delivered by administering nucleic acid molecules which comprise nucleotide sequences that encode the protein that is expressed to produce the protein when the nucleic acid molecules are taken up by cells. According in addition embodiments of the invention in which the IL-8 and RANTES proteins are each delivered by administering the protein themselves, some embodiments include delivery of nucleic acid molecules that encode the proteins in addition to and/or instead of delivery of the proteins themselves. In some embodiments, nucleotide sequences that encode both proteins are on a single nucleic acid molecule. In some embodiments, compositions comprise two nucleic acid molecule in which the nucleotide sequences that encode one protein is on one nucleic acid molecule and the nucleotide sequences that encode the other protein is on another nucleic acid molecule. In some embodiments, compositions comprise two nucleic acid molecules in which the nucleotide sequences that encodes one protein is on one nucleic acid molecule and the nucleotide sequences that encode both proteins are on another nucleic acid molecule.

The present invention further relates to compositions for delivering the immunomodulating proteins and methods of using the same. Aspects of the present invention relate to nucleic acid molecules that comprise a nucleotide sequence that encodes IL-8 operably linked to regulatory elements and/or a nucleotide sequence that encodes RANTES operably linked to regulatory elements. The present invention further relates to injectable pharmaceutical compositions which comprise such nucleic acid molecules.

The nucleic acid molecules that comprise a nucleotide sequence that encodes IL-8 operably linked to regulatory elements and/or a nucleotide sequence that encodes RANTES operably linked to regulatory elements may be delivered using any of several well known technologies including DNA injection (also referred to as DNA vaccination), recombinant vectors such as recombinant adenovirus, recombinant adenovirus associated virus and recombinant vaccinia.

DNA vaccines are described in U.S. Pat. Nos. 5,593,972, 5,739,118, 5,817,637, 5,830,876, 5,962,428, 5,981,505, 5,580,859, 5,703,055, 5,676,594, and the priority applications cited therein, which are each incorporated herein by reference. In addition to the delivery protocols described in those applications, alternative methods of delivering DNA are described in U.S. Pat. Nos. 4,945,050 and 5,036,006, which are both incorporated herein by reference.

Routes of administration include, but are not limited to, intramuscular, intranasally, intraperitoneal, intradermal, subcutaneous, intravenous, intraarterially, intraoccularly and oral as well as topically, transdermally, by inhalation or suppository or to mucosal tissue such as by lavage to vaginal, rectal, urethral, buccal and sublingual tissue. Preferred routes of administration include to mucosal tissue, intramuscular, intraperitoneal, intradermal and subcutaneous injection. Genetic constructs may be administered by means including, but not limited to, traditional syringes, needleless injection devices, or “microprojectile bombardment gene guns”.

When taken up by a cell, the genetic construct(s) may remain present in the cell as a functioning extrachromosomal molecule and/or integrate into the cell's chromosomal DNA. DNA may be introduced into cells where it remains as separate genetic material in the form of a plasmid or plasmids. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be administered to the cell. It is also contemplated to provide the genetic construct as a linear minichromosome including a centromere, telomeres and an origin of replication. Gene constructs may remain part of the genetic material in attenuated live microorganisms or recombinant microbial vectors which live in cells. Gene constructs may be part of genomes of recombinant viral vaccines where the genetic material either integrates into the chromosome of the cell or remains extrachromosomal.

Genetic constructs include regulatory elements necessary for gene expression of a nucleic acid molecule. The elements include: a promoter, an initiation codon, a stop codon, and a polyadenylation signal. In addition, enhancers are often required for gene expression of the sequence that encodes the target protein or the immunomodulating protein. It is necessary that these elements be operable linked to the sequence that encodes the desired proteins and that the regulatory elements are operably in the individual to whom they are administered.

Initiation codons and stop codon are generally considered to be part of a nucleotide sequence that encodes the desired protein. However, it is necessary that these elements are functional in the individual to whom the gene construct is administered. The initiation and termination codons must be in frame with the coding sequence.

Promoters and polyadenylation signals used must be functional within the cells of the individual.

Examples of promoters useful to practice the present invention, especially in the production of a genetic vaccine for humans, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metalothionein.

Examples of polyadenylation signals useful to practice the present invention, especially in the production of a genetic vaccine for humans, include but are not limited to SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, is used.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV.

Genetic constructs can be provided with mammalian origin of replication in order to maintain the construct extrachromosomally and produce multiple copies of the construct in the cell. Plasmids pCEP4 and pREP4 from Invitrogen (San Diego, Calif.) contain the Epstein Barr virus origin of replication and nuclear antigen EBNA-1 coding region which produces high copy episomal replication without integration.

In some preferred embodiments related to immunization applications, nucleic acid molecule(s) are delivered which include nucleotide sequences that encode a target protein, the immunomodulating protein and, additionally, genes for proteins which further enhance the immune response against such target proteins. Examples of such genes are those which encode other cytokines and lymphokines such as α-interferon, gamma-interferon, platelet derived growth factor (PDGF), TNF, epidermal growth factor (EGF), IL-1, IL-2, IL-4, IL-6, IL-10 and IL-12. In some embodiments, it is preferred that the gene for GM-CSF is included in genetic constructs used in immunizing compositions.

An additional element may be added which serves as a target for cell destruction if it is desirable to eliminate cells receiving the genetic construct for any reason. A herpes thymidine kinase (tk) gene in an expressible form can be included in the genetic construct. The drug gangcyclovir can be administered to the individual and that drug will cause the selective killing of any cell producing tk, thus, providing the means for the selective destruction of cells with the genetic construct.

In order to maximize protein production, regulatory sequences may be selected which are well suited for gene expression in the cells the construct is administered into. Moreover, codons may be selected which are most efficiently transcribed in the cell. One having ordinary skill in the art can produce DNA constructs which are functional in the cells.

One method of the present invention comprises the steps of administering nucleic acid molecules intramuscularly, intranasally, intraperatoneally, subcutaneously, intradermally, or topically or by lavage to mucosal tissue selected from the group consisting of inhalation, vaginal, rectal, urethral, buccal and sublingual.

In some embodiments, the nucleic acid molecule is delivered to the cells in conjunction with administration of a polynucleotide function enhancer or a genetic vaccine facilitator agent. Polynucleotide function enhancers are described in U.S. Ser. No. 08/008,342 filed Jan. 26, 1993, U.S. Ser. No. 08/029,336 filed Mar. 11, 1993, U.S. Ser. No. 08/125,012 filed Sep. 21, 1993, and International Application Serial Number PCT/US94/00899 filed Jan. 26, 1994, which are each incorporated herein by reference. Genetic vaccine facilitator agents are described in U.S. Ser. No. 08/221,579 filed Apr. 1, 1994, which is incorporated herein by reference. The co-agents which are administered in conjunction with nucleic acid molecules may be administered as a mixture with the nucleic acid molecule or administered separately simultaneously, before or after administration of nucleic acid molecules. In addition, other agents which may function transfecting agents and/or replicating agents and/or inflammatory agents and which may be co-administered with a GVF include growth factors, cytokines and lymphokines such as α-interferon, gamma-interferon, platelet derived growth factor (PDGF), TNF, epidermal growth factor (EGF), IL-1, IL-2, IL-4, IL-6, IL-10 and IL-12 as well as fibroblast growth factor, surface active agents such as immune-stimulating complexes (ISCOMS), Freund's incomplete adjuvant, LPS analog including monophosphoryl Lipid A (MPL), muramyl peptides, quinone analogs and vesicles such as squalene and squalene, and hyaluronic acid may also be used administered in conjunction with the genetic construct. In some embodiments, an immunomodulating protein may be used as a GVF.

The pharmaceutical compositions according to the present invention comprise about 1 nanogram to about 2000 micrograms of DNA. In some preferred embodiments, pharmaceutical compositions according to the present invention comprise about 5 nanogram to about 1000 micrograms of DNA. In some preferred embodiments, the pharmaceutical compositions contain about 10 nanograms to about 800 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 25 to about 250 micrograms of DNA. In some preferred embodiments, the pharmaceutical compositions contain about 100 to about 200 micrograms DNA.

The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vaso-constriction agent is added to the formulation.

According to some embodiments of the invention, methods of inducing immune responses against an immunogen are provided by delivering a combination of the immunogen, IL-8 and RANTES to an individual. According to some embodiments of the invention, the agents for delivering IL-8 and RANTES, either as a protein or a nucleic acid molecule encoding the protein, are administered as a component of or otherwise as a supplement to in conjunction with a vaccine composition. The vaccine may be either a subunit vaccine, a killed vaccine, a live attenuated vaccine, a cell vaccine, a recombinant vaccine or a nucleic acid or DNA vaccine. In the case of a live attenuated vaccine, a cell vaccine, a recombinant vaccine or a nucleic acid or DNA vaccine, the IL-8 and RANTES may be encoded by the nucleic acid molecules of these vaccines. Alternatively or additionally, the IL-8 and/or RANTES protein may be used as an adjuvant.

According to some embodiments, the immunogen, IL-8 and RANTES may each, independently, be delivered directly in protein form and/or as nucleic acid molecules which comprise nucleotide sequences that encode the protein operably linked to regulatory elements necessary for expression in the individual. When the nucleic acid molecules are taken up by cells of the individual, the nucleotide sequences that encode the protein are expressed in the cells and the protein is thereby delivered to the individual.

As set forth below, methods of delivering the immunogen, IL-8 and RANTES may be accomplished by delivery of gene constructs that encode one of the immunogen, IL-8 and RANTES, respectively. As discussed below, the immunogen may be referred to as a target protein. In embodiments which do not include administration of an immunogen, the description below may be carried out without the provision or delivery of gene constructs that encode the target protein.

The present invention is useful to elicit broad immune responses against a target protein, i.e. proteins specifically associated with pathogens, allergens or the individual's own “abnormal” cells. The present invention is useful to immunize individuals against pathogenic agents and organisms such that an immune response against a pathogen protein provides protective immunity against the pathogen. The present invention is useful to combat hyperproliferative diseases and disorders such as cancer by eliciting an immune response against a target protein that is specifically associated with the hyperproliferative cells. The present invention is useful to combat autoimmune diseases and disorders by eliciting an immune response against a target protein that is specifically associated with cells involved in the autoimmune condition.

According to some aspects of the present invention, DNA or RNA that encodes a target protein and immunomodulating proteins is introduced into the cells of tissue of an individual where it is expressed, thus producing the encoded proteins. The DNA or RNA sequences encoding the target protein and one or both immunomodulating proteins are linked to regulatory elements necessary for expression in the cells of the individual. Regulatory elements for DNA expression include a promoter and a polyadenylation signal. In addition, other elements, such as a Kozak region, may also be included in the genetic construct.

In some embodiments, expressible forms of sequences that encode the target protein and expressible forms of sequences that encode both immunomodulating proteins are found on the same nucleic acid molecule that is delivered to the individual. In some embodiments, expressible forms of sequences that encode the target protein occur on a separate nucleic acid molecule from the nucleic acid molecules that contain expressible forms of sequences that encode one or both immunomodulating proteins. In some embodiments, expressible forms of sequences that encode the target protein and expressible forms of sequences that encode one of the immunomodulatory proteins occur on a one nucleic acid molecule that is separate from the nucleic acid molecule that contain expressible forms of sequences that encode the other of the two immunomodulating proteins. In such cases, both molecules are delivered to the individual. In some embodiments, expressible forms of sequences that encode the target protein occur on separate nucleic acid molecule from the nucleic acid molecules that contain expressible forms of sequences that encode both immunomodulating proteins. In such cases, both molecules are delivered to the individual. In some embodiments, expressible forms of sequences that encode the target protein occur on separate nucleic acid molecule from the nucleic acid molecules that contain expressible forms of sequences that encode one of the two immunomodulating proteins which occur on separate nucleic acid molecule from the nucleic acid molecules that contain expressible forms of sequences that encode the other of the two immunomodulating proteins. In such cases, all three molecules are delivered to the individual. In addition, any combination of one, two or three DNA molecules encoding one, two or three proteins can be delivered to produce a multitude of combinations with a multitude of numbers of different molecules. Importantly, copies of the coding sequences for the target protein, RANTES protein and IL-8 are provided in at least one nucleic acid molecule.

The nucleic acid molecule(s) may be provided as plasmid DNA, the nucleic acid molecules of recombinant vectors or as part of the genetic material provided in an attenuated vaccine or cell vaccine. Alternatively, in some embodiments, the target protein and/or wither or both immunomodulating proteins may be delivered as a protein in addition to the nucleic acid molecules that encode them or instead of the nucleic acid molecules that encode them.

Genetic constructs may comprise a nucleotide sequence that encodes a target protein or an immunomodulating protein operably linked to regulatory elements needed for gene expression. According to the invention, combinations of gene constructs which include one that comprises an expressible form of the nucleotide sequence that encodes a target protein and one that includes an expressible form of the nucleotide sequence that encodes an immunomodulating protein are provided. Incorporation into a living cell of the DNA or RNA molecule(s) which include the combination of gene constructs results in the expression of the DNA or RNA and production of the target protein and the immunomodulating protein. An enhanced immune response against the target protein results.

The present invention may be used to immunize an individual against all pathogens such as viruses, prokaryote and pathogenic eukaryotic organisms such as unicellular pathogenic organisms and multicellular parasites. The present invention is particularly useful to immunize an individual against those pathogens which infect cells and which are not encapsulated such as viruses, and prokaryote such as gonorrhoea, listeria and shigella. In addition, the present invention is also useful to immunize an individual against protozoan pathogens which include a stage in the life cycle where they are intracellular pathogens. Table 1 provides a listing of some of the viral families and genera for which vaccines according to the present invention can be made. DNA constructs that comprise DNA sequences which encode the peptides that comprise at least an epitope identical or substantially similar to an epitope displayed on a pathogen antigen such as those antigens listed on the tables are useful in vaccines. Moreover, the present invention is also useful to immunize an individual against other pathogens including prokaryotic and eukaryotic protozoan pathogens as well as multicellular parasites such as those listed on Table 2.

In order to produce a genetic vaccine to protect against pathogen infection, genetic material which encodes immunogenic proteins against which a protective immune response can be mounted must be included in a genetic construct as the coding sequence for the target. Whether the pathogen infects intracellularly, for which the present invention is particularly useful, or extracellularly, it is unlikely that all pathogen antigens will elicit a protective response. Because DNA and RNA are both relatively small and can be produced relatively easily, the present invention provides the additional advantage of allowing for vaccination with multiple pathogen antigens. The genetic construct used in the genetic vaccine can include genetic material which encodes many pathogen antigens. For example, several viral genes may be included in a single construct thereby providing multiple targets.

Tables 1 and 2 include lists of some of the pathogenic agents and organisms for which genetic vaccines can be prepared to protect an individual from infection by them. In some preferred embodiments, the methods of immunizing an individual against a pathogen are directed against HIV, HTLV or HBV.

Another aspect of the present invention provides a method of conferring a broad based protective immune response against hyperproliferating cells that are characteristic in hyperproliferative diseases and to a method of treating individuals suffering from hyperproliferative diseases. Examples of hyperproliferative diseases include all forms of cancer and psoriasis.

It has been discovered that introduction of a genetic construct that includes a nucleotide sequence which encodes an immunogenic “hyperproliferating cell”—associated protein into the cells of an individual results in the production of those proteins in the vaccinated cells of an individual. To immunize against hyperproliferative diseases, a genetic construct that includes a nucleotide sequence which encodes a protein that is associated with a hyperproliferative disease is administered to an individual.

In order for the hyperproliferative-associated protein to be an effective immunogenic target, it must be a protein that is produced exclusively or at higher levels in hyperproliferative cells as compared to normal cells. Target antigens include such proteins, fragments thereof and peptides which comprise at least an epitope found on such proteins. In some cases, a hyperproliferative-associated protein is the product of a mutation of a gene that encodes a protein. The mutated gene encodes a protein which is nearly identical to the normal protein except it has a slightly different amino acid sequence which results in a different epitope not found on the normal protein. Such target proteins include those which are proteins encoded by oncogenes such as myb, myc, fyn, and the translocation gene bcr/abl, ras, src, P53, neu, trk and EGRF. In addition to oncogene products as target antigens, target proteins for anti-cancer treatments and protective regimens include variable regions of antibodies made by B cell lymphomas and variable regions of T cell receptors of T cell lymphomas which, in some embodiments, are also used target antigens for autoimmune disease. Other tumor-associated proteins can be used as target proteins such as proteins which are found at higher levels in tumor cells including the protein recognized by monoclonal antibody 17-1A and folate binding proteins.

While the present invention may be used to immunize an individual against one or more of several forms of cancer, the present invention is particularly useful to prophylactically immunize an individual who is predisposed to develop a particular cancer or who has had cancer and is therefore susceptible to a relapse. Developments in genetics and technology as well as epidemiology allow for the determination of probability and risk assessment for the development of cancer in individual. Using genetic screening and/or family health histories, it is possible to predict the probability a particular individual has for developing any one of several types of cancer.

Similarly, those individuals who have already developed cancer and who have been treated to remove the cancer or are otherwise in remission are particularly susceptible to relapse and reoccurrence. As part of a treatment regimen, such individuals can be immunized against the cancer that they have been diagnosed as having had in order to combat a recurrence. Thus, once it is known that an individual has had a type of cancer and is at risk of a relapse, they can be immunized in order to prepare their immune system to combat any future appearance of the cancer.

The present invention provides a method of treating individuals suffering from hyperproliferative diseases. In such methods, the introduction of genetic constructs serves as an immunotherapeutic, directing and promoting the immune system of the individual to combat hyperproliferative cells that produce the target protein.

The present invention provides a method of treating individuals suffering from autoimmune diseases and disorders by conferring a broad based protective immune response against targets that are associated with autoimmunity including cell receptors and cells which produce “self”-directed antibodies.

T cell mediated autoimmune diseases include Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dernatomyositis, psoriasis, vasculitis, Wegener's granulomatosis, Crohn's disease and ulcerative colitis. Each of these diseases is characterized by T cell receptors that bind to endogenous antigens and initiate the inflammatory cascade associated with autoimmune diseases. Vaccination against the variable region of the T cells would elicit an immune response including CTLs to eliminate those T cells.

In RA, several specific variable regions of T cell receptors (TCRs) which are involved in the disease have been characterized. These TCRs include Vβ-3, Vβ-14, Vβ-17 and Vα-1 7. Thus, vaccination with a DNA construct that encodes at least one of these proteins will elicit an immune response that will target T cells involved in RA. See: Howell, M. D., et al., 1991 Proc. Natl. Acad. Sci. USA 88:10921-10925; Paliard, X., et al., 1991 Science 253:325-329; Williams, W. V., et al., 1992 J. Clin. Invest. 90:326-333; each of which is incorporated herein by reference.

In MS, several specific variable regions of TCRs which are involved in the disease have been characterized. These TCRs include Vβ-7 and Vα-10. Thus, vaccination with a DNA construct that encodes at least one of these proteins will elicit an immune response that will target T cells involved in MS. See: Wucherpfennig, K. W., et al., 1990 Science 248:1016-1019; Oksenberg, J. R., et al., 1990 Nature 345:344-346; each of which is incorporated herein by reference.

In scleroderma, several specific variable regions of TCRs which are involved in the disease have been characterized. These TCRs include Vβ-6, Vβ-8, Vβ-14 and Vα-16, Vα-3C, Vα-7, Vα-14, Vα-15, Vα-16, Vα-28 and Vα-12. Thus, vaccination with a DNA construct that encodes at least one of these proteins will elicit an immune response that will target T cells involved in scleroderma.

In order to treat patients suffering from a T cell mediated autoimmune disease, particularly those for which the variable region of the TCR has yet to be characterized, a synovial biopsy can be performed. Samples of the T cells present can be taken and the variable region of those TCRs identified using standard techniques. Genetic vaccines can be prepared using this information.

B cell mediated autoimmune diseases include Lupus (SLE), Grave's disease, myasthenia gravis, autoimmune hemolytic anemia, autoimmune thrombocytopenia, asthma, cryoglobulinemia, primary biliary sclerosis and pernicious anemia. Each of these diseases is characterized by antibodies which bind to endogenous antigens and initiate the inflammatory cascade associated with autoimmune diseases. Vaccination against the variable region of antibodies would elicit an immune response including CTLs to eliminate those B cells that produce the antibody.

In order to treat patients suffering from a B cell mediated autoimmune disease, the variable region of the antibodies involved in the autoimmune activity must be identified. A biopsy can be performed and samples of the antibodies present at a site of inflammation can be taken. The variable region of those antibodies can be identified using standard techniques. Genetic vaccines can be prepared using this information.

In the case of SLE, one antigen is believed to be DNA. Thus, in patients to be immunized against SLE, their sera can be screened for anti-DNA antibodies and a vaccine can be prepared which includes DNA constructs that encode the variable region of such anti-DNA antibodies found in the sera.

Common structural features among the variable regions of both TCRs and antibodies are well known. The DNA sequence encoding a particular TCR or antibody can generally be found following well known methods such as those described in Kabat, et al. 1987 Sequence of Proteins of Immunological Interest U.S. Department of Health and Human Services, Bethesda Md., which is incorporated herein by reference. In addition, a general method for cloning functional variable regions from antibodies can be found in Chaudhary, V. K., et al., 1990 Proc. Natl. Acad. Sci. USA 87:1066, which is incorporated herein by reference.

In addition to using expressible forms of immunomodulating protein coding sequence to improve genetic vaccines, the present invention relates to improved attenuated live vaccines and improved vaccines which use recombinant vectors to deliver foreign genes that encode antigens. Examples of attenuated live vaccines and those using recombinant vectors to deliver foreign antigens are described in U.S. Pat. Nos. 4,722,848; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; and 5,482,713, which are each incorporated herein by reference. Gene constructs are provided which include the nucleotide sequence that encodes an immunomodulating protein is operably linked to regulatory sequences that can function in the vaccinee to effect expression. The gene constructs are incorporated in the attenuated live vaccines and recombinant vaccines to produce improved vaccines according to the invention.

The present invention provides an improved method of immunizing individuals that comprises the step of delivering gene constructs to the cells of individuals as part of vaccine compositions which include are provided which include DNA vaccines, attenuated live vaccines and recombinant vaccines. The gene constructs comprise a nucleotide sequence that encodes an immunomodulating protein and that is operably linked to regulatory sequences that can function in the vaccinee to effect expression. The improved vaccines result in an enhanced cellular immune response.

### EXAMPLE

### DNA Vaccines Encoding Chemokines IL-8 and RANTES Drive Antigen-Specific Th1 Type Immune Responses and Enhance Protective Immunity Against Herpes Simplex Virus-2 In Vivo

### Introduction

The initiation of immune or inflammatory reactions is a complex process involving the coordinated expression of costimulatory molecules, adhesion molecules, cytokines, and chemokines. In particular, chemokines are important in the molecular regulation of trafficking of immune cells to the peripheral sites of host defenses. The chemokine superfamily consists of two subfamilies based upon the presence (α family) or absence (β family) of a single amino acid sequence separating two cysteine residues. α and β chemokines have been shown to induce direct migration of various immune cell types, including neutrophils, eosinophils, basophils, and monocytes. The α chemokine family (CXC type), interleukin (IL)-8 and interferon-γ inducible protein (IP)-10, and the β chemokine family (CC type), RANTES (regulated on activation, normal T cell expressed and secreted), monocyte chemotactic protein (MCP-1) and macrophage inflammatory protein (MIP)-1α have been shown to chemoattract T lymphocytes. In particular, IL-8 and IP-10 have been known to chemoattract neutrophils, inducing them to leave the bloodstream and migrate into the surrounding tissues. Similarly, RANTES chemoattracts monocytes, unstimulated CD4+/CD45RO+ memory T cells and stimulated CD4+ and CD8+ T cells. MIP-1α has been known to chemoattract and degranulate eosinophils. MIP-1α also induces histamine release from basophils and mast cells and chemoattracts basophils and B cells. MCP-1 is an important chemokine in chronic inflammatory disease. MCP-1 induces monocytes to migrate from the bloodstream to become tissue macrophages. MCP-1 also chemoattracts T lymphocytes of the activated memory subset. Recent studies support that chemokine receptors mark T cell subsets and that chemokines may be involved in the generation of antigen-specific immune responses.

As reported herein, the DNA vaccine model was utilized to investigate whether chemokines could modulate immune responses and then impact protection from herpes simplex virus (HSV)-2 challenge in a defined mouse model system. To investigate the modulation of immune responses and protective immunity, a DNA expression construct encoding HSV-2 protein was co-delivered with the gene plasmids encoding for chemokines (IL-8, IP-10, RANTES, MCP-1, MIP-1α). The modulatory effects in antigen-specific immune induction and protection from challenge was then analyzed. Coinjection with IL-8 and RANTES was observed to enhance antigen-specific immune responses and protection from HSV challenge. On the other hand, coinjection with IP-10, MCP-1, MIP-1α had overall detrimental effects on the protection status. These studies support that chemokines can act and modulate important immune responses and disease progression in a manner reminiscent of cytokines. Significant immune modulation could be achieved through the use of codelivered chemokine cDNAs, impacting not just an immune response but also disease protection. Furthermore, use of chemokine gene-delivered adjuvants, in particular IL-8 and RANTES could be important in crafting more efficacious vaccines or in immune therapies for HSV.

### Methods

Mice.

Female 4- to 6-week-old BALB/c mice were purchased from Harlan Sprague-Dawley (Indianapolis, Ind.). They were cared for under the guidelines of the National Institutes of Health (Bethesda, Md.) and the University of Pennsylvania IACUC (Philadelphia, Pa.).

Reagents.

HSV-2 strain 186 (a kind gift from P. Schaffer, University of Pennsylvania, Philadelphia, Pa.) was propogated in the Vero cell line (American Type Culture Collection, Rockville, Md.). The DNA vaccine, pAPL-gD2 (pgD) encoding HSV-2 gD protein was previously described in Pachuk, C. J. et al., “Humoral and cellular immune responses to herpes simplex virus-2 glycoprotein D generated by facilitated DNA immunization of mice” Current topics Microbiol. Immunol. 1998 226:79-89 which is incorporated herein by reference. The expression vectors, pCDNA3-IL-8, pCDNA3-IP-10, pCDNA3-RANTES, pCDNA3-MCP-1, pCDNA3-MIP-1α, pCDNA3-TNF-α, and pCDNA3-TNF-β were previously constructed as described in Kim, J. J. et al., “CD8 positive T-cells influence antigen-specific immune responses through the expression of chemokines” J. Clin. Invest. 1998 102:1112-1124 and Kim, J. J. et al. “Modulation of amplitude and direction of in vivo immune responses by co-administration of cytokine gene expression cassettes with DNA immunogens” Eur. J. Immunol. 1998 28:1089-1103, which are incorporated herein by reference. Plasmid DNA was produced in bacteria and purified by double banded CsCl preparations. Recombinant HSV-2 gD proteins, a generous gift from G. H. Cohen and R. J. Eisenberg, University of Pennsylvania, Philadelphia, Pa., were used as recombinant antigens in these studies.

DNA Inoculation of Mice.

The quadriceps muscles of BALB/c mice were injected with gD DNA constructs formulated in 100 μl of phosphate-buffered saline and 0.25% bupivacaine-HCl (Sigma, St. Louis, Mo.) via a 28-gauge needle (Becton Dickinson, Franklin Lakes, N.J.). Samples of various chemokine and cytokine gene expression cassettes were mixed with gpD plasmid solution prior to injection.

ELISA.

Enzyme-linked immunosorbent assay (ELISA) was performed as previously described in Sin, J. I. et al. “In vivo modulation of vaccine-induced immune responses toward a Th1 phenotype increases potency and vaccine effectiveness in a herpes simplex virus type 2 mouse model” J. Virol. 1999 73: 501-509 and Sin, J. I. et al. “Enhancement of protective humoral (Th2) and cell-mediated (Th1) immune responses against herpes simplex virus-2 through co-delivery of granulocyte macrophage-colony stimulating factor expression cassettes” Eur. J. Immunol. 1998 28:3530-3540 which are incorporated herein by reference. In particular, for the determination of relative levels of gD-specific IgG subclasses, anti-murine IgG1, IgG2a, IgG2b, or IgG3 conjugated with HRP (Zymed, San Francisco, Calif.) were substituted for anti-murine IgG-HRP. The ELISA titers were determined as the reverse of the highest sera dilution showing the same optical density as sera of naive mice.

T Helper (Th) Cell Proliferation Assay.

Th cell proliferation assay was performed as previously described Sin, J. I. et al. J. Virol. 1999 supra and Sin, J. I. et al. Euro. J. Immunol. 1998 supra. The isolated cell suspensions were resuspended to a concentration of 1×10⁶ cells/ml. A 100 μl aliquot containing 1×10⁵ cells was immediately added to each well of a 96 well microtiter flat bottom plate. HSV-2 gD protein at the final concentration of 1 μg/ml and 5 μg/ml was added to wells in triplicate. The cells were incubated at 37° C. in 5% CO₂ for three days. One μCi of tritiated thymidine was added to each well and the cells were incubated for 12 to 18 hours at 37° C. The plate was harvested and the amount of incorporated tritiated thymidine was measured in a Beta Plate reader (Wallac, Turki, Finland). Stimulation Index was determined from the formula:

Stimulation Index (SI)=(experimental count-spontaneous count)/spontaneous count

Spontaneous count wells include 10% fetal calf serum which serves as irrelevant protein control. To assure that cells were healthy, 5 μg/ml PHA (Sigma) was used as a polyclonal stimulator positive control.

Th1 and Th2 Type Cytokines and Chemokines.

A 1 ml aliquot containing 6×10⁶ splenocytes was added to wells of 24 well plates. Then, 1 μg of HSV-2 gD protein/ml was added to each well. After 2 days incubation at 37° C. in 5% CO₂, cell supernatants were secured and then used for detecting levels of IL-2, IL-10, IFN-γ, RANTES, MCP-1 and MIP-1α using commercial cytokine and chemokine kits (Biosource, Intl., Camarillo, Calif. and R&D Systems, Minneapolis, Md.) by adding the extracellular fluids to the cytokine or chemokie-specific ELISA plates.

Intravaginal HSV-2 Challenge.

Mice were challenged as previously described with some modification Milligan, G. N. et al. “Analysis of herpes simplex virus-specific T cells in the murine female genital tract following genital infection with herpes simplex virus type 2” Virol. 1995 212:481-489 and McDermott, et al. “Immunity in the female genital tract after intravaginal vaccination of mice with an attenuated strain of herpes simplex virus type 2” J. Virol. 1984 51:747-753 which are incorporated herein by reference. Before inoculating the virus, the intravaginal area was swabbed with a cotton tipped applicator (Hardwood Products Company, Guiford, Me.) soaked with 0.1 M NaOH solution and then cleaned with dried cotton applicators. Mice were then examined daily to evaluate pathological conditions and survival rates.

Statistical Analysis.

Statistical analysis was done using the paired Student's test. Values between different immunization groups were compared. p values<0.05 were considered significant.

### Results

Co-Administration of Chemokine Plasmids Influences Systemic IgG Production

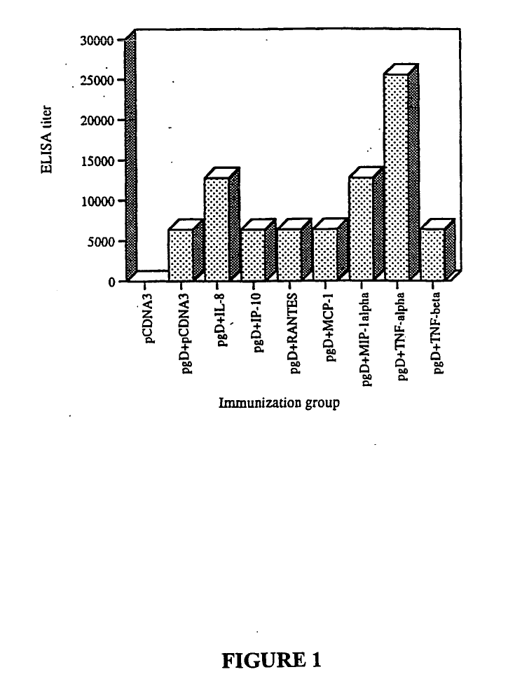

The in vivo effects of selected chemokines on the induction of antigen-specific antibody responses were first investigated. As controls animals were immunized with gD vaccine and 2 proinflammatory cytokines, TNF family genes (TNF-α and TNF-β). These proinflammatory cytokines were studied as they are thought to similarly be involved in early immune responses and should serve as positive controls based upon previously generated data. As shown in FIG. 1, ELISA titers of equally pooled sera collected 2 weeks post the second immunization were determined as 12,800 (IL-8), 6,400 (IP-10), 6,400 (RANTES), 6,400 (MCP-1), 12,800 (MIP-1a), TNF-α (25,600), TNF-β (6,400) and 6,400 (for the gD DNA vaccine alone). This shows that coinjection with either IL-8 and MIP-1α genes results in a moderate, but not significant enhancement of gD-specific IgG antibodies. In contrast, IP-10, RANTES or MCP-1 showed similar levels of antibody responses to that of pgD vaccination alone. The TNF-α cDNA control resulted in systemic IgG levels significantly higher than those of gD DNA vaccine alone exactly as previously observed.

Coimmunization with Chemokine Plasmids Shifts IgG Subclasses to Th1 or Th2 Isotypes

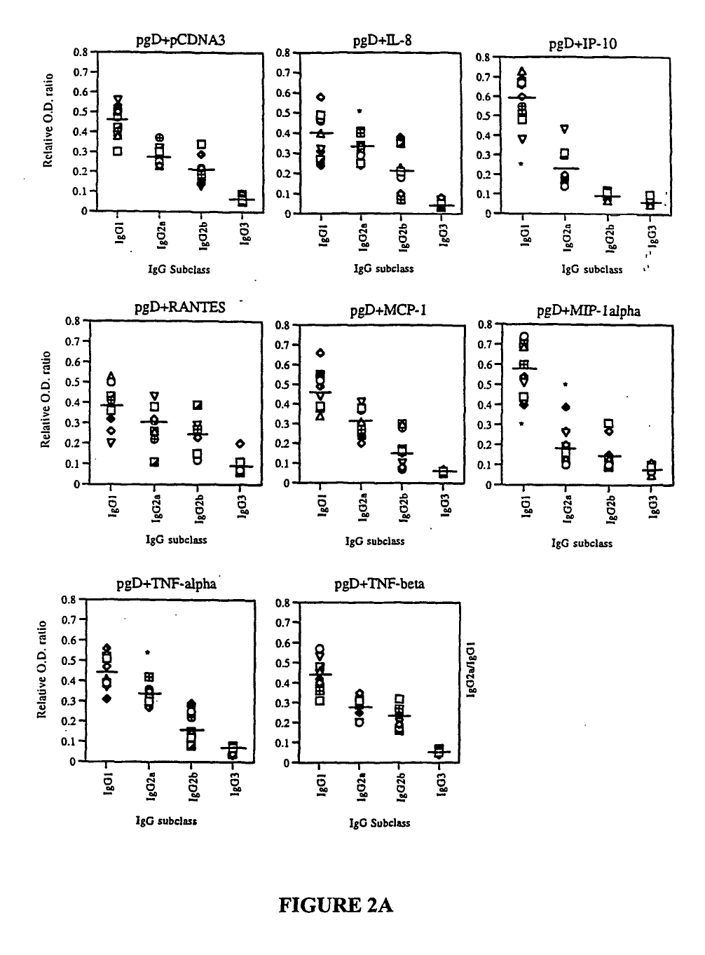

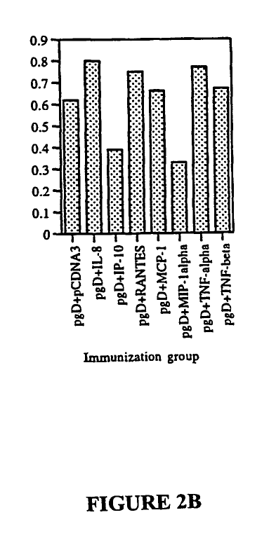

IgG subclasses give an indication of the Th1 vs Th2 nature of the induced immune responses. The IgG subclasses induced by the coinjections were analyzed. IgG isotypes induced by each immunization group are shown in FIGS. 2A and the relative ratios of IgG2a to IgG1 (Th1 to Th2) are shown in FIG. 2B. The pgD immunized group had a IgG2a to IgG1 ratio of 0.62. Coinjection with either IL-8, RANTES or TNF-α genes increased the relative ratio of gD-specific IgG2a to IgG1 to 0.8. On the other hand, coinjection with either IP-10 or MIP-1α decreased the relative ratio of IgG2 to IgG1 (0.3 and 0.4), whereas co-immunization with either MCP-1 or TNF-β genes resulted in an IgG subtype pattern similar to pgD vaccination alone. This analysis supports a conclusion that IL-8 and RANTES drive immune responses towards Th1 phenotype in vivo in a similar manner to γ-IFN type cytokines.

IL-8 and RANTES Coinjections Enhance Th Cell Proliferative Responses

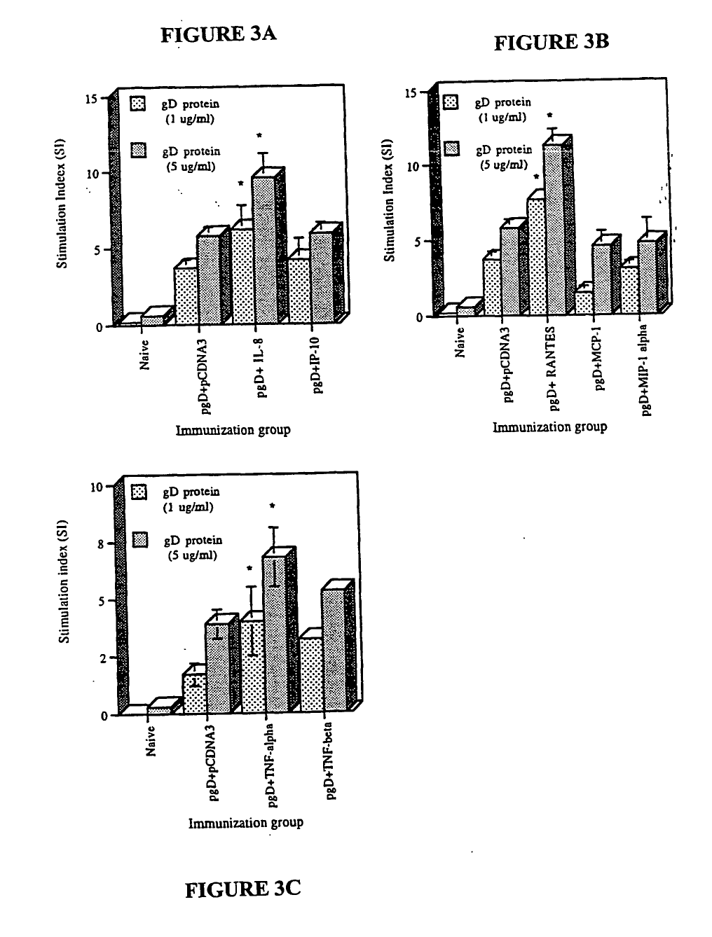

The cell proliferation is a standard parameter used to evaluate the potency of cell-mediated immunity. Th cell proliferative responses following coimmunization with cytokine genes were measured by stimulating splenocytes from immunized animals in vitro with gD proteins. As shown in FIGS. 3A-3C, pgD DNA vaccination alone resulted in gD-specific Th cell proliferative responses. Significant enhancement of Th cell proliferative responses over that of gD DNA vaccine alone were observed with coinjection with either IL-8, RANTES or TNF-α cDNAs. A slight enhancement in proliferation was observed by coinjection with TNF-β genes. In contrast, coimmunization with either IP-10, MCP-1 or MIP-1α genes appeared to have minimal effects on the levels of Th cell proliferative responses. However, the coinjections showed no effects on PHA-induced non-specific Th cell proliferative responses (S.I. range was 40 to 50). The gD plasmid vaccination does not result in CTL responses due to a lack of CTL epitope in the Balb/c background. However, to evaluate cellular effects in more detail cytokine production profiles was examined.

Chemokine Coinjections Influence Production of Th1 and Th2 Cytokines

Th1 cytokines (IL-2 and IFN-γ) and Th2 cytokines (IL-4, IL-5 and IL-10) have been a mainstay in the understanding of the polarization of immune responses. Th1 immune responses are thought to drive induction of cellular immunity, whereas Th2 immune responses preferentially drive humoral immunity. Based on the IgG phenotype results, the Th1 vs Th2 issue was further evaluated by analyzing cytokine release directly. As shown in Table 3, IL-2 production was dramatically increased almost 7 fold by coinjection with IL-8 cDNA. IL-2 was also induced by coinjection with TNF-α cDNA, and by coinjection with the MIP-1α cassette. In particular, production of IFN-γ was most significantly enhanced by codelivery of RANTES, 20 fold and IL-8, 6 fold, further supporting the isotyping results and demonstrating that IL-8 and RANTES mediate Th1 type cellular immune responses in an antigen-dependent fashion. RANTES, IL-8, TNF-α and TNF-β coinjections each also enhanced IL-10 production significantly higher than pgD vaccine alone. This illustrates that IL-8 and RANTES drive T cells of predominantly Th1 over a Th2 type.

Chemokine Coinjections Influence Production of β Chemokines

To determine if chemokine coinjection could induce β chemokine production in an antigen-dependent manner, animals were coimmunized and release levels of β chemokines of splenocytes were analyzed after in vitro stimulation with recombinant gD antigen or control antigen. As shown in Table 4, MCP-1 production was dramatically increased by coinjection with IL-8 cDNA, but was decreased by coinjection with RANTES and MIP-1α cassettes. In particular, production of MIP-1α is most significantly enhanced by codelivery of RANTES and IL-8. In the case of RANTES, IL-8 and RANTES coinjections enhanced RANTES production higher than pgD vaccine alone. This indicates that RANTES modulates antigen-specific immune responses differently from IL-8 in the HSV model. This also supports that chemokines modulate their own production.

IL-8 and RANTES Chemokine Coinjections Enhance Protection from Intravaginal HSV-2 Challenge

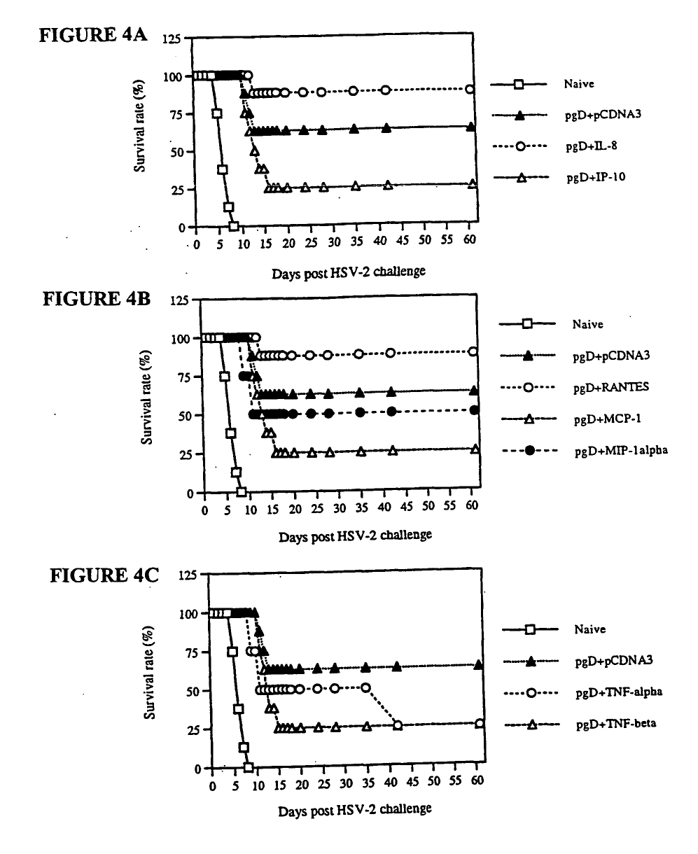

It is important that antigen-specific immune modulation influences pathogen's replication. Protective efficacy of chemokine coinjection was analyzed in the murine herpes challenge model. Mice were coimmunized i.m. with DNA vectors at 0 and 2 weeks and then challenged with HSV-2 at 3 weeks post second immunization. Intravaginal challenge route was chosen as HSV-2 infects mucocutaneously. As shown in FIGS. 4A and 4B, immunization with gD DNA vaccine (60 μg per mouse) alone resulted in 63% of survival of mice from intravaginal challenge with 200 LDSO of HSV-2. Coinjection with IL-8 and RANTES cDNA increased the survival rate to 88%, an almost 30% enhancement of protection rate, whereas coinjection with 40 μg of MCP-1 and IP-10 decreased the survival rate to 25%, an approximately 40% decrease in the overall protection rate. Similarly, MIP-1α coinjection also resulted in a decrease in the survival rate. This supports the conclusion that chemokines IL-8 and RANTES enhanced protection from HSV-2 infection through antigen-specific immune modulation. TNF family coinjection worsens protection from intravaginal HSV-2 challenge The protective efficacy of TNF family coinjection was compared in the herpes challenge model. Coinjection with TNF-α and TNF-β cDNA resulted in decreased survival to 25%, a 40% decrease in the protection rate (FIG. 4C). This data further supports that coinjection of some immunomodulatory cDNA will lower vaccine effectiveness, supporting that the quality of the response is particularly important.

### Discussion

HSV is the causative agent of a spectrum of human diseases, such as cold sores, ocular infections, encephalitis, and genital infections. HSV can establish viral latency with frequent recurrences in the host. During viral infection, neutralizing antibody inactivates viral particles, but is unable to control intracellular HSV infection. Rather, cellular-mediated immunity is a major effector function which kills HSV-infected cells. The ability of B cell-suppressed mice to control primary HSV infection or the ability of adoptively transferred T cells to prevent subsequent viral infection further suggests that cell-mediated immunity might be directly related in inhibition of viral infection and its spread. It also has been well documented that both CD4+ and CD8+ T cells are involved in prevention of HSV infection. Furthermore, there have been several reports that Th1 type CD4+ T cells play a more crucial role for protection from HSV-2 challenge. When CD4+ T cells were depleted in vivo, protective immunity against HSV was lost. Moreover, Th1 type CD4+ T cells generate a large amount of IFN-γ. IFN-γ upregulates class I and class II expression on HSV-infected cells to allow better recognition by cytotoxic CD4+ T cells and CD8+ CTL, and has direct anti-HSV effects. Codelivery with Th1 type cytokine cDNAs worsen disease status. Similarly, protection enhanced by codelivering with a prototypic Th1 type cytokine IL-12 cDNA was mediated Th1 type CD4+ T cells in HSV challenge model, underscoring the importance of Th1 type T cell-mediated protective immunity against HSV infection.

In animal models, immunization of some HSV glycoproteins or DNA constructs expressing specific viral components provide complete or partial protection against viral challenge. Several HSV viral proteins have been analyzed as potential immunization targets. Immunization with cDNA encoding the gC, ICP27 or gD proteins has been shown to induce antigen-specific immune responses and protection against in vivo challenge with HSV in animals. Recently, clinical trials using a subunit vaccine failed to protect from recurrent HSV infection, supporting that additional insight is needed to design a more effective approach for this pathogen.

The in vivo effects of selected chemokines on the induction of protective immunity against HSV-2 infection was investigated by coinjecting them as plasmid cassettes along with gD DNA vaccine constructs. Groups coimmunized with IL-8 and MIP-1α chemokine genes had slightly higher IgG responses than the gD immunized group in a similar manner to TNF-α as a vaccine adjuvant. Furthermore, modulation of antigen-specific IgG isotype responses has been achieved by using chemokines as molecular adjuvants. IL-8 and RANTES significantly increased the relative ratio of gD-specific IgG2a to IgG1, as compared to gD DNA vaccine alone or coinjection with MCP-1 or with the TNF-β control. However, coinjection with IP-10 and MIP-1α genes induced more favorable production of IgG1, as compared to IgG2a. Thus, these results extend prior findings in the HIV model that the shift in humoral immune responses to either Th1 or Th2 could be modulated by chemokines, again suggesting that chemokines can modulate cytokine production in vivo.

In vitro immune parameters, such as Th cell proliferative and CTL responses have been used to evaluate the potency of cell-mediated immunity. Only plasmid coinjection with IL-8 and RANTES induced higher Th cell proliferation in a similar manner to the TNF-α control. IL-8 coimmunization also resulted in increased production of IL-2 and INF-γ significantly higher than gD DNA vaccine alone, further supporting the isotyping results and demonstrate that IL-8 mediates Th1 type cellular immune responses in an antigen-dependent fashion. IL-8 coinjection also enhanced MCP-1, MIP-1 and RANTES production, indicating that IL-8 can modulate β chemokine production in vivo. RANTES coinjection resulted in increased production of IFN-γ, IL-10, MIP-1α, and RANTES, but decreased production of IL-2 and MCP-1. This indicates that RANTES modulates antigen-specific immune responses differently from IL-8 in the HSV model.

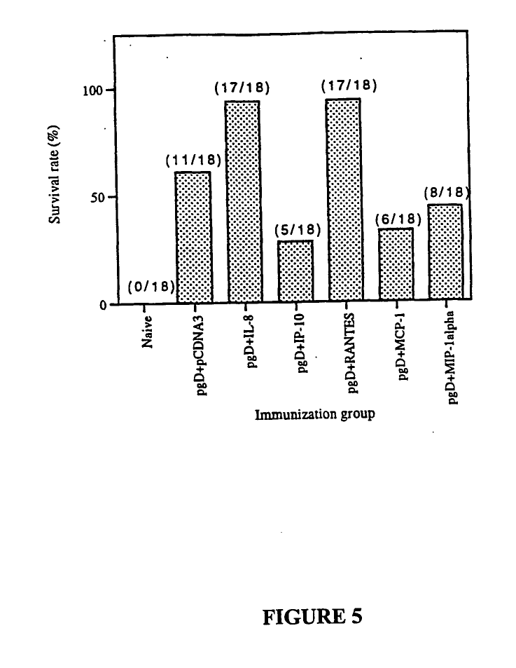

In HSV challenge studies, gD vaccination alone showed 63% survival rates at the challenge inoculum of 200 LD₅₀ of HSV-2. By co-injecting chemokine IL-8 and RANTES cDNAs, better survival rates (88%) and less severe herpetic lesion formation were achieved. In contrast, codelivery of chemokine genes (IP-10 and MCP-1) reduced the rate of survival of challenged mice to 25%, more than a 50% reduction in overall survival from the gD vaccine alone. Similarly, MIP-1α coinjection also negatively influenced the survival rate of vaccinated animals. These observations are striking if one considers the total number of animals tested in each chemokine group (survival rates of gD alone, 13 of 18 [72%]; survival rates of IL-8, 17 of 18 [94%]′ survival rates of IP-10, 7 of 18 [39%]; survival rates of MIP-1α, 10 of 18 [56%]) (FIG. 5). This indicates that coinjection with IL-8 and RANTES chemokine gene enhances protection from lethal HSV challenge while coinjection with IP-10 and MCP-1 and to a less degree MIP-1α make animals more susceptible to viral infection in spite of the induction of immune responses. Coinjection with Th1 type cytokine gene enhances protection rate from lethal HSV challenge while Th2 type cytokine coinjection increases susceptibility of animal to viral infection. In pathogenesis studies, the importance of Th1 like cytokine response for resistance from pathogenic infection has been reported. Thus, it seems likely that Th1 and/or Th2 type immune responses are being driven by these chemokines, resulting in an impact on protection from HSV infectious challenge based on the quality of the immune responses.

In the case of TNF family, coinjection with both TNF-α and TNF-β genes also reduced the rate of survival of challenged mice to 25%, more than 50% reduction in overall survival from the gD vaccine alone. Although gD-specific antibody and Th cell proliferation levels as well as cytokine production levels (IL-2, IFN-γ, IL-10) of mice coinjected with TNF-α genes were much higher than those of gD DNA vaccination alone, TNF cytokine-mediated susceptibility to HSV-2 infection was observed in those animals. The reason for this observation is unclear but strongly supports that the quality of the responses is significantly important for controlling pathogenic infection.

In conclusion, the data demonstrate that chemokines could modulate immune responses to Th1 and/or Th2 types in an antigen-dependent fashion. Such activities have been previously only associated with cytokines. These data imply that chemokines have as central a role as cytokines in the induction of antigen-specific immunity. This finding broadens our weapons for infectious diseases. Furthermore, the use of chemokines to modulate immune responses for cancer therapies should also be considered.

TABLE 1

| | |
|---|---|
| Picornavirus Family | |
| Genera: | Rhinoviruses: (Medical) responsible for ˜50% cases of the common |
| | cold. |
| | Etheroviruses: (Medical) includes polioviruses, |
| | coxsackieviruses, echoviruses, and human enteroviruses |
| | such as hepatitis A virus. |
| | Apthoviruses: (Veterinary) these are the foot and mouth |
| | disease viruses. |
| Target antigens: | VP1, VP2, VP3, VP4, VPG |
| Calcivirus Family | |
| Genera: | Norwalk Group of Viruses: (Medical) these viruses are an |
| | important causative agent of epidemic gastroenteritis. |
| Togavirus Family | |
| Genera: | Alphaviruses: (Medical and Veterinary) examples include |
| | Senilis viruses, RossRiver virus and Eastern & Western |
| | Equine encephalitis. |
| | Reovirus: (Medical) Rubella virus. |
| Flariviridue Family | |
| | Examples include: (Medical) dengue, yellow fever, |
| | Japanese encephalitis, St. Louis encephalitis and tick borne |
| | encephalitis viruses. |

Hepatitis C Virus: (Medical) these viruses are not placed in a family yet but are believed
to be either a togavirus or a flavivirus. Most similarity is with togavirus family.

| | |
|---|---|
| Coronavirus Family: | |
| (Medical and Veterinary) | |
| | Infectious bronchitis virus (poultry) |
| | Porcine transmissible gastroenteric virus (pig) |
| | Porcine hemagglutinating encephalomyelitis virus (pig) |
| | Feline infectious peritonitis virus (cats) |
| | Feline enteric coronavirus (cat) |
| | Canine coronavirus (dog) |
| | The human respiratory coronaviruses cause ˜40 cases of |
| | common cold. EX. 224E, 0C43 |
| | Note - coronaviruses may cause non-A, B or C hepatitis |
| Target antigens: | E1 - also called M or matrix protein |
| | E2 - also called S or Spike protein |
| | E3 - also called HE or hemagglutin-elterose |
| | glycoprotein (not present in all coronaviruses) |
| | N - nucleocapsid |
| Rhabdovirus Family | |
| Genera: | Vesiliovirus |
| | Lyssavirus: (medical and veterinary) |
| | rabies |
| Target antigen: | G protein |
| | N protein |
| Filoviridue Family: | |
| (Medical) | |
| | Hemorrhagic fever viruses such as Marburg and Ebola |
| | virus |
| Paramyxovirus Family: | |
| Genera: | Paramyxovirus: (Medical and Veterinary) |
| | Mumps virus, New Castle disease virus (important |
| | pathogen in chickens) |
| | Morbillivirus: (Medical and Veterinary) |
| | Measles, canine distemper |
| | Pneuminvirus: (Medical and Veterinary) |
| | Respiratory syncytial virus |
| Orthomyxovirus Family | |
| (Medical) | |
| | The Influenza virus |
| Bungavirus Family | |
| Genera: | Bungavirus: (Medical) California encephalitis, LA Crosse |
| | Phlebovirus: (Medical) Rift Valley Fever |
| | Hantavirus: Puremala is a hemahagin fever virus |
| | Nairvirus (Veterinary) Nairobi sheep disease |
| | Also many unassigned bungaviruses |
| Arenavirus Family | |
| (Medical) | |
| | LCM, Lassa fever virus |
| Reovirus Family | |
| Genera: | Reovirus: a possible human pathogen |
| | Rotavirus: acute gastroenteritis in children |
| | Orbiviruses: (Medical and Veterinary) |
| | Colorado Tick fever, Lebombo (humans) equine |
| | encephalosis, blue tongue |
| Retrovirus Family | |
| Sub-Family: | Oncorivirinal: (Veterinary) (Medical) feline leukemia |
| | virus, HTLVI and HTLVII |
| | Lentivirinal: (Medical and Veterinary) HIV, feline |
| | immunodeficiency virus, equine infections, anemia virus |
| | Spumavirinal |
| Papovavirus Family | |
| Sub-Family: | Polyomaviruses: (Medical) BKU and JCU viruses |
| Sub-Family: | Papillomavirus: (Medical) many viral types associated |
| | with cancers or malignant progression of papilloma |
| Adenovirus (Medical) | |
| | EX AD7, ARD., O.B. - cause respiratory disease - some |
| | adenoviruses such as 275 cause enteritis |
| Parvovirus Family | |
| (Veterinary) | |
| | Feline parvovirus: causes feline enteritis |
| | Feline panleucopeniavirus |
| | Canine parvovirus |
| | Porcine parvovirus |
| Herpesvirus Family | |
| Sub-Family: | alphaherpesviridue |
| Genera: | Simplexvirus (Medical) |
| | HSVI, HSVII |
| | Varicellovirus: (Medical - Veterinary) pseudorabies - |
| | varicella zoster |
| Sub-Family - | betaherpesviridue |
| Genera: | Cytomegalovirus (Medical) |
| | HCMV |
| | Muromegalovirus |
| Sub-Family: | Gammaherpesviridue |
| Genera: | Lymphocryptovirus (Medical) |
| | EBV - (Burkitts lympho) |
| | Rhadinovirus |
| Poxvirus Family | |
| Sub-Family: | Chordopoxviridue (Medical - Veterinary) |
| Genera: | Variola (Smallpox) |
| | Vaccinia (Cowpox) |
| | Parapoxivirus - Veterinary |
| | Auipoxvirus - Veterinary |
| | Capripoxvirus |
| | Leporipoxvirus |
| | Suipoxvirus |
| Sub-Family: | Entemopoxviridue |
| Hepadnavirus Family | |
| | Hepatitis B virus |
| Unclassified | Hepatitis delta virus |

TABLE 2

| | |
|---|---|
| Bacterial pathogens | Pathogenic gram-positive cocci include: pneumococcal; staphylococcal; |
| | and streptococcal. Pathogenic gram-negative cocci include: |
| | meningococcal; and gonococcal. |
| | Pathogenic enteric gram-negative bacilli include: enterobacteriaceae; |
| | pseudomonas, acinetobacteria and eikenella; melioidosis; salmonella; |
| | shigellosis; hemophilus; chancroid; brucellosis; tularemia; yersinia |
| | (pasteurella); streptobacillus moniliformis and spirillum; listeria |
| | monocytogenes; erysipelothrix rhusiopathiae; diphtheria; cholera; |
| | anthrax; donovanosis (granuloma inguinale); and bartonellosis. |
| | Pathogenic anaerobic bacteria include: tetanus; botulism; other clostridia; |
| | tuberculosis; leprosy; and other mycobacteria. Pathogenic spirochetal |
| | diseases include: syphilis; treponematoses: yaws, pinta and endemic |
| | syphilis; and leptospirosis. |
| | Other infections caused by higher pathogen bacteria and pathogenic fungi |
| | include: actinomycosis; nocardiosis; cryptococcosis, blastomycosis, |
| | histoplasmosis and coccidioidomycosis; candidiasis, aspergillosis, and |
| | mucormycosis; sporotrichosis; paracoccidiodomycosis, petriellidiosis, |
| | torulopsosis, mycetoma and chromomycosis; and dermatophytosis. |
| | Rickettsial infections include rickettsial and rickettsioses. |
| | Examples of mycoplasma and chlamydial infections include: mycoplasma |
| | pneumoniae; lymphogranuloma venereum; psittacosis; and perinatal |
| | chlamydial infections. |
| Pathogenic eukaryotes | Pathogenic protozoans and helminths and infections thereby include: |
| | amebiasis; malaria; leishmaniasis; trypanosomiasis; toxoplasmosis; |
| | pneumocystis carinii; babesiosis; giardiasis; trichinosis; filariasis; |
| | schistosomiasis; nematodes; trematodes or flukes; and cestode |
| | (tapeworm) infections. |

TABLE 3
Production levels of IL-2, IL-10 and IFN-γ of splenocytes after in
vitro gD stimulation^{a}

| Immunization | IL-2 | IFN-γ | IL-10 |
|---|---|---|---|
| group | (pg/ml) | (pg/ml) | (pg/ml) |
| Naive | 16.7 ± 0.8 | 10.5 ± 0.7 | 17.1 ± 6.12 |
| pgD + pCDNA3 | 134.7 ± 3.5 | 22.4 ± 2.4 | 57.1 ± 4.4 |
| pgD + IL8 | 756.4 ± 5.4 | 138.5 ± 4.7 | 128 ± 13 |
| pgD + IP-10 | 143.5 ± 3.9 | 31.5 ± 2.5 | 69.9 ± 1.9 |
| pgD + RANTES | 59.9 ± 1.1 | 520 ± 13 | 360 ± 46.5 |
| pgD + MCP-1 | 93.6 ± 4.7 | 17.9 ± 0.5 | 49.7 ± 2.3 |
| pgD + MIP-1α | 345.4 ± 18 | 55.4 ± 1.8 | 22 ± 2.1 |
| pgD + TNF-α | 403 ± 13.3 | 77 ± 6.3 | 86.8 ± 6.2 |
| pgD + TNF-β | 288 ± 5.6 | 20.8 ± 1.5 | 78.3 ± 3.6 |
| ^{a}Each group of mice (n = 2) was immunized with gD DNA vaccines (60 μg per mouse) plus chemokine genes (40 μg per mouse) or TNF cDNAs (40 μg per mouse) at 0 and 2 weeks. Two weeks after the last DNA injection, two mice were sacrificed and spleen cells were pooled. Splenocytes were stimulated with 1 μg of gD proteins/ml for 2 days. Samples were assayed in triplicate and the values represent means of released cytokine | | | |
| #concentrations ± standard deviation. This represents one of three separate experiments showing a similar result. | | | |

TABLE 4
Production levels of MCP-1, MIP-1α and RANTES of splenocytes
after in vitro gD stimulation^{a}.

| Immunization | MCP-1 | MIP-1α | RANTES |
|---|---|---|---|
| group | (pg/ml) | (pg/ml) | (pg/ml) |
| Naive | 153.8 ± 5.7 | 247 ± 11 | 769 ± 7 |
| pgD + pCDNA3 | 234 ± 5.3 | 747 ± 39 | 917 ± 55 |
| pgD + IL-8 | 322 ± 24 | 1,411 ± 113 | 1,284 ± 53 |
| pgD + IP-10 | 246.3 ± 2.7 | 1,407 ± 459 | 831 ± 52 |
| pgD + RANTES | 189.7 ± 0 | 2,267 ± 219 | 1,077 ± 32 |
| gD + MCP-1 | 209.2 ± 6.4 | 725 ± 501 | 646 ± 45 |
| pgD + MIP-1α | 142.7 ± 3.3 | 787 ± 94 | 690 ± 39 |
| ^{a}Each group of mice (n = 2) was immunized with gD DNA vaccines (60 μg per mouse) plus chemokine genes (40 μg per mouse) at 0 and 2 weeks. Two weeks after the last DNA injection, two mice were sacrificed and spleen cells were pooled. Splenocytes were stimulated with 1 μg of gD proteins/ml for 2 days. Samples were assayed in triplicate and the values represent means of released chemokine concentrations ± standard | | | |
| #deviation. This represents one of three separate experiments showing a similar result. | | | |

1. A composition comprising:
isolated RANTES protein and/or a nucleic acid molecule that encodes RANTES protein; and
isolated IL-8 protein and/or a nucleic acid molecule that encodes IL-8 protein.

2. The composition of claim 1 further comprising a target protein and/or a nucleic acid molecule that encodes a target protein.

3. The composition of claim 1 comprising a plasmid comprising a nucleotide sequence that encodes an IL-8 operably linked to regulatory elements and a nucleotide sequence that encodes RANTES operably linked to regulatory elements.

4. The composition of claim 3 wherein said plasmid further comprises a nucleotide sequence that encodes an immunogen operably linked to regulatory elements,

5. The composition of claim 4 wherein said immunogen is a pathogen antigen, a cancer-associated antigen or an antigen linked to cells associated with autoimmune diseases.

6. The composition of claim 4 wherein said immunogen is a pathogen antigen.

7. The composition of claim 4 wherein said immunogen is a herpes simplex antigen.

8. The composition of claim 7 wherein said herpes simplex antigen is HSV2gD.

9. An injectable pharmaceutical composition comprising the composition of claims 1-8.

10. A method of inducing an immune response in an individual against an immunogen comprising administering to said individual
isolated RANTES protein and/or a nucleic acid molecule that encodes RANTES protein;
isolated IL-8 protein and/or a nucleic acid molecule that encodes IL-8 protein; and
a target protein and/or a nucleic acid molecule that encodes a target protein.

11. The method of claim 10 comprising administering to said individual
a nucleic acid molecule that encodes RANTES protein;
a nucleic acid molecule that encodes IL-8 protein; and
a nucleic acid molecule that encodes a target protein.

12. The method of claim 11 comprising administering to said individual a plasmid comprising a nucleotide sequence that encodes RANTES protein, a nucleotide sequence that encodes IL-8 protein and a nucleotide sequence that encodes a target protein.

13. The method of claim 11 comprising administering to said individual two or more different plasmids wherein said two or more different plasmids collectively comprise a nucleotide sequence that encodes RANTES protein, a nucleotide sequence that encodes IL-8 protein and a nucleotide sequence that encodes a target protein.

14. The method of any one of claims 11-13 comprising administering to said individual RANTES protein and/or IL-8 protein and/or a target protein.

15. The method of claim 10-14 wherein said target protein is a pathogen antigen, a cancer-associated antigen or an antigen linked to cells associated with autoimmune diseases.

16. The method of claim 10-15 wherein said immunogen is a pathogen antigen.

17. The method of any one of claims 10-16 wherein said target protein is a herpes simplex virus antigen.

18. The method of any one of claims 10-17 wherein said target protein is herpes simplex virus antigen HSV2gD.

19. A method of modulating an individual's immune system comprising administering to said individual
isolated RANTES protein and/or a nucleic acid molecule that encodes RANTES protein; and
isolated IL-8 protein and/or a nucleic acid molecule that encodes IL-8 protein.

20. The method of claim 19 comprising administering to said individual
a nucleic acid molecule that encodes RANTES protein;
a nucleic acid molecule that encodes IL-8 protein.

21. The method of claim 20 comprising administering to said individual a plasmid comprising a nucleotide sequence that encodes RANTES protein and a nucleotide sequence that encodes IL-8 protein.

22. The method of claim 20 comprising administering to said individual two or more different plasmids wherein said two or more different plasmids collectively comprise a nucleotide sequence that encodes RANTES protein and a nucleotide sequence that encodes IL-8 protein.

23. The method of any one of claims 19-22 comprising administering to said individual RANTES protein and/or IL-8 protein.

24. A recombinant vaccine comprising a nucleotide sequence that encodes an immunogen operably linked to regulatory elements, a nucleotide sequence that encodes IL-8, and a nucleotide sequence that encodes RANTES.

25. The recombinant vaccine of claim 24 wherein said immunogen is a pathogen antigen, a cancer-associated antigen or an antigen linked to cells associated with autoimmune diseases.

26. The recombinant vaccine of claim 24 wherein said immunogen is a pathogen antigen.

27. A method of inducing an immune response in an individual against an immunogen comprising administering to said individual a recombinant vaccine of claim 24.

28. The recombinant vaccine of claim 24 wherein said recombinant vaccine is a recombinant vaccinia vaccine.

29. A live attenuated pathogen comprising a nucleotide sequence that encodes IL-8 and a nucleotide sequence that encodes RANTES.

30. A method of immunizing an individual against a pathogen comprising administering to said individual the live attenuated pathogen of claim 29.
